(19) United States
(12) Patent Application Publication
(10) Pub. No.: US 2017/0205404 A1
(43) Pub. Date: Jul. 20, 2017

(54) MULTIFUNCTIONAL BEADS AND METHODS OF USE FOR CAPTURING RARE CELLS

(71) Applicant: GENERAL ELECTRIC COMPANY, Schenectady, NY (US)

(72) Inventors: Christopher Michael Puleo, Niskayuna, NY (US); Ernest William Kovacs, Cohoes, NY (US); Brian Michael Davis, Niskayuna, NY (US)

(21) Appl. No.: 15/366,520

(22) Filed: Dec. 1, 2016

Related U.S. Application Data
(60) Provisional application No. 62/280,244, filed on Jan. 19, 2016.

(51) Int. Cl. G01N 33/543; G01N 33/569

(52) U.S. Cl. CPC G01N 33/54326; G01N 33/56972

(57) ABSTRACT

Described are multi-functional beads and methods to capture rare cells directly from low-volume biological samples and perform both functional and genomic assays from those cells. This is accomplished using a multifunctional capture bead that allows co-localization of both the single cell capture element and the molecular assay components. When combined with a digital microfluidic platform this enables encoding and/or barcoding of specific single cells.

### CROSS-REFERENCE TO RELATED APPLICATIONS

This application is a non-provisional of and claims priority from U.S. provisional application, Ser. No. 62/280,244, filed Jan. 19, 2016, the entire disclosure of which is incorporated herein by reference.

### FEDERAL SUPPORT CLAUSE

This invention was made with Government support under contract number 1U24AI118667-01 awarded by the National Institutes of Health. The Government has certain rights in the invention.

### BACKGROUND

Current methods of selecting specific cells from a mixed population require large sample volumes and many cells. These techniques include MACS (magnetic activated cell sorting) and FACS (fluorescence activated cell sorting). This is due to the need to capture specific cells and then transfer them to follow-on assays. Cell loss associated with manipulation of the sorted sample remains high, and rare cells and/or cells from very small sample volumes are lost.

Other techniques such as single cell analysis in digital microfluidic devices, enables direct injection of single cell suspensions, bar-coding, and highly parallel analysis of single cells. However, these devices do not function with “raw” samples, and in order to function cells must be purified and diluted in fresh buffer. These sample preparation steps cause loss of rare cells/samples, and negate the efficiencies of direct injection into the microfluidic device. Therefore, while analytical tools have kept pace with massively parallel and multi-parameter analytical techniques, sample preparation techniques have not.

### BRIEF DESCRIPTION

Described are multi-functional beads that are labeled with both a cell capture element and a biomolecular capture element that allows direct injection of a cell into a digital microfluidic assay colocalized with at least one reagent necessary for a biological assay. This solves the current mismatch between highly parallel/multi-parameter microfluidic analytical devices and the sample preparation required to inject rare/low-volume raw biological samples.

In one embodiment, a method of performing an assay on rare cells captured from a biological sample is provided comprising contacting a solution containing the biological sample with a multifunctional bead. The multifunctional bead comprises a microsphere between 0.1 and 100 μm in size, a cell capture element, on the surface of the microsphere, capable of binding to a protein or cell specific marker on the surface of a rare cell, and a biomolecular capture element, on the surface of the microsphere, capable of binding to biomolecular components contained within or produced by the rare cell. The method involves incubating the multifunctional beads with a biological sample containing the rare cells and binding the multifunctional bead to the rare cells through the surface capture element to create a bead-bound rare cells. Biomolecules are captured which are contained within or produced by the rare cells through the biomolecular capture element which may be assayed by analyzing the biomolecules captured.

In one embodiment, a method of using the bead bound to a rare cell is disclosed. The method involves flowing the solution containing the bead-bound rare cells through a microfluidic device, the microfluidic device having microfluidic compartments and partitioning the bead-bound rare cells of the solution into at least one of the microfluidic compartments. The method further comprises contacting a biomolecular capture element with bead-bound rare cells, and capturing biomolecules contained within or produced by the rare cells through the biomolecular capture element.

Also disclosed is a multi-functional bead comprising a microsphere between 0.1 and 100 μm in size, a cell capture element, on the surface of the microsphere, capable of binding to a protein or cell specific marker on the surface of a rare cell, and a biomolecular capture element, on the surface of the microsphere, capable of binding to biomolecular components contained within or produced by the rare cell.

### BRIEF DESCRIPTION OF DRAWINGS

These and other features, aspects, and advantages of the present invention will become better understood when the following detailed description is read with reference to the accompanying drawings in which like characters represent like parts throughout the drawings.

FIG. 1 is an example of a multifunctional bead/particle.

FIG. 2a depicts an illustrative example of microfluidic digitization of a large volume sample into microchambers.

FIG. 2b depicts an illustrative example of microfluidic digitization of a large volume sample in microdroplets.

FIGS. 3a and 3b are a graphical representation of the limitation of Poisson loading into microfluidic digital systems; 3A using microchambers and 3B using microfluidic droplets.

FIG. 4 is a flow diagram of a method of using digital microfluidic platforms with multifunctional beads for deterministic single cell loading and assays showing process steps A-D.

FIG. 5a is an illustrative example of a method of using digital microfluidic platforms and multi-functional beads; shown is an example a magnetic ratchet used to separate cells from a bulk sample and trap them individually on magnetic pillars.

FIG. 5b is an illustrative example of a method of use of the devices of FIGS. 5a and 5b; isolating the cells in place by placing a microchamber array over the top of the device or using the magnetic ratchet to move the cell toward an isolation chamber.

FIG. 6a shows representative data of cytokine binding function on a bead conjugated to a pMHC II tetramer.

FIG. 6b is an illustration showing function of the cytokine capture element in binding recombinant cytokine (IFN).

FIG. 6c a graphical representation of the results of cytokine binding test with a lowest measured value of 133 pg per bead.

FIGS. 7a-7c are histograms showing function of individual multi-functional beads for rare cell capture and analysis; FIG. 7a is APC, FIG. 7b is PE, and FIG. 7c is FITC.

FIGS. 8a and 8b are graphical representation of cyctokine secretion and capture from T cells captured in microwells; FIG. 8a is a graphical representation of mean fluorescence intensity, FIG. 8b is a histogram of average fluorescence intensity.

### DETAILED DESCRIPTION

Extraction of rare target cells from biological samples remains one of the key requirements for modern diagnostics and cell biology research. New techniques such as Drop-Seq have ushered in methods for parallel analysis of many single cells (transcriptomes). However, these techniques still require relatively large starting volumes and cell numbers (recommendation is 100 cells/uL). As such there still exist major constraints in designing mechanism of action studies in clinical trials for which the biological samples collected contain rare and limited cell numbers.

Further, restricted amounts of tissue, cells and fluids are common in many clinical studies, including samples collected from pediatric or immunocompromised patients. Novel, multi-parameter, sample sparing assays are needed to obtain maximal information from limited amounts of biological materials, which may also be referred to as a biological sample. As used herein a biological sample may include, but is not limited to a cells suspension from a blood or tissue sample such as a biopsy. These samples may be harvested from a blood drawn, a needle aspirate, biopsy sampling, or any body site tissue specimen.

To respond to the needs of improved methods for rare cell capture, in certain embodiments, multifunctional beads or particles are provided that contain both cell capture elements, for example antibodies or tetramers specific to cell surface proteins, and cellular assay elements for example antibodies, nucleic acids, or molecules to capture specific targets of a biomolecular assay. The multifunctional beads are capable of to first capturing cells and subsequently undergoing at least the first biomolecular reaction/step in a biomolecular assay. In certain embodiments, the reaction occurs within a microfluidic device or microscale liquid compartments. The process may be used to ensure that each captured cell is co-localized with the reagents necessary to initiate the biomolecular reaction. The unique multifunctional bead is able to be used to drive deterministic loading, loading specific cells to the microchambers, and to provide access to the compartmentalized cell for post-capture processing.

In certain embodiments, tools and methods to specifically capture rare cells directly from low-volume biological samples are described and used to perform both functional and genomic assays from those cells. This is accomplished using a multifunctional capture bead that allows co-localization of both the single cell capture element and the molecular assay components. In combination, with a digital microfluidic platform, this enables encoding and/or barcoding of specific single cells. In certain embodiments, the assay may also include quantitating a specific cell type in the biological sample such that the number of the specific cells may be counted or estimated. In addition to immunophenotyping, the method may also be used in of genomes and transcriptomes, as well as antibody discovery, HLA typing, haplotyping and drug discovery.

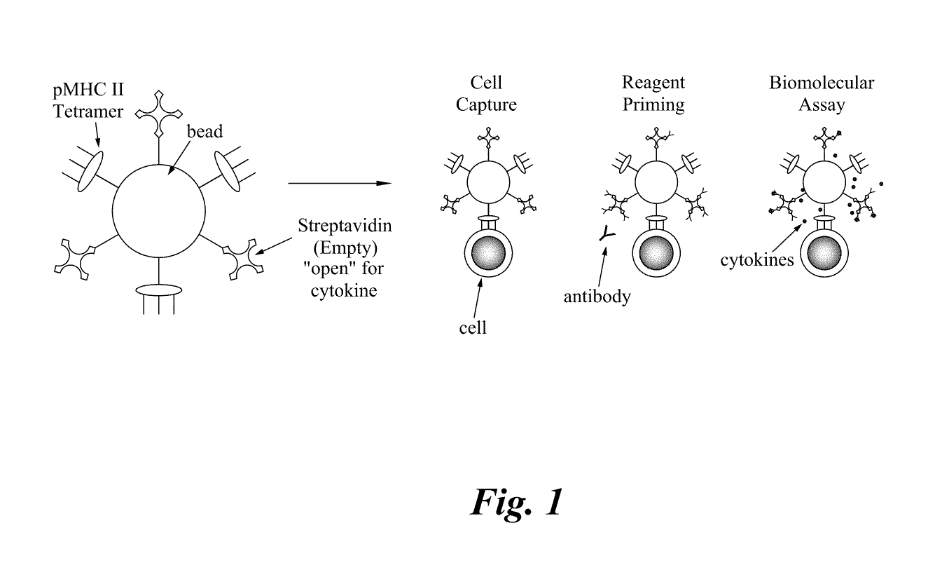

A non-limiting example of a method using a multifunctional bead is provided in FIG. 1. In the example shown, a bead is conjugated with both pMHC II tetramers, having a specific antigenic peptide sequence, and with streptavidin binding sites. Singly bound pMHC II tetramers are capable of binding to antigen specific T cells while streptavidin is a common reagent in biomolecular assay for binding to biotin labeled assay reagents. As such, the bead is conjugated with two binding sites rendering the bead multi-functional. The bead is capable of cell capture, through one site, and capable of priming with a biomolecular assay reagent, on the other site. This effectively provides a microfluidic compartment for co-localization of a specific cell and reagent. In certain embodiments, the specific cell may be considered a rare cell, that is a cell that occurs in limited quantities or at low concentrations in the cell population.

In a further step of the process, a biomolecular assay reagent may be used which, is capable of binding to a site on the bead. One example of such a reagent, as shown in FIG. 1 is a biotinylated antibody which can capture a targeted molecule. In the example the captured or targeted molecule is a cytokine proteins secreted by immune cells. Here the molecule is a specific marker on the cell surface that functionally is an antigen, capable of complexing with the biomolecular assay reagent which is an antibody.

As shown in FIG. 1, the biotinylated antibodies first bind to the streptavidin on the multifunctional bead; providing a site for cytokine capture

Thus, the doubly labelled beads, functionalized with cell capture MHC tetramers and cytokine antibodies are now multifunctional; enabling cell capture and priming with a biomolecular assay reagent. The bead may function as a microfluidic compartment, as a site for-localization of a specific cell and reagent.

In certain embodiments, beads may be used to capture various molecules. Examples of a captured molecule include, but are not limited to, secreted cytokines, proteins, or intracellular nucleic acids and proteins after lysis. Other examples of a biomolecular assay reagent or molecular binding element include, but are not limited to artificially synthesized bioactive polymers, peptide tetramers, antibodies, nucleic acids and oligonucleotides, fluorescent conjugates for optical analysis or metal conjugates for mass spectrometry analysis, or combinations thereof.

In certain other embodiments, cell capture and priming with a biomolecular assay reagent provides a method of co-localization of a specific cell and reagent into a microfluidic compartment. In certain embodiments, microfluidic digitization may be used, by allowing priming of a bead bind site with cell specific assay components, after compartmentalization, without contamination from the molecular components of other cells.

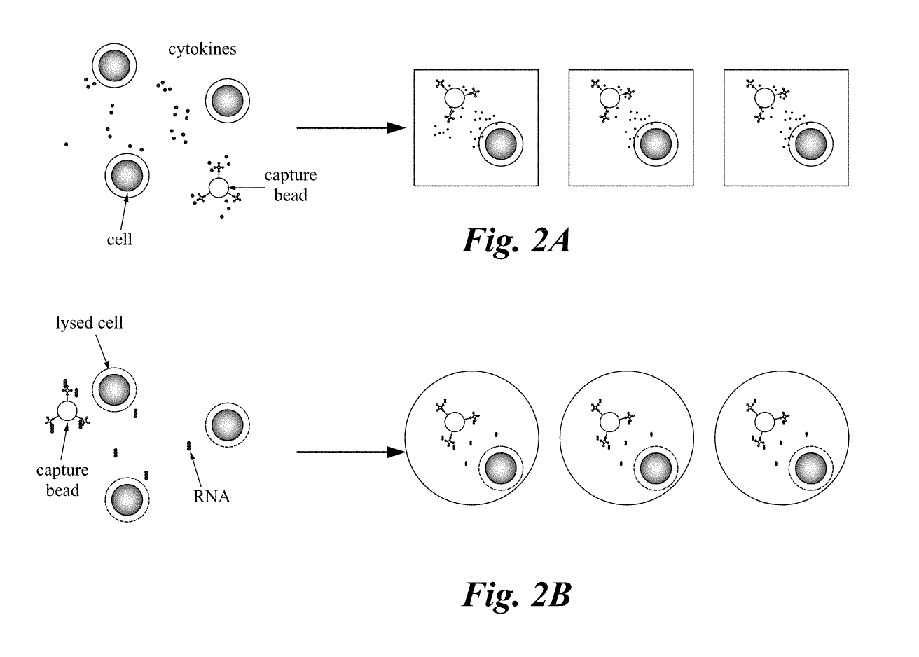

The need for this type of approach is illustrated in FIGS. 2a and 2b showing the limitations of using a single cell assays using singly functional beads; FIG. 2a is using a microchamber device, while FIG. 2b is representative of microdroplets. In brief, digitization or compartmentalization of a cell with a molecular capture bead allows capture of cell specific product/components without contamination from neighboring cells. The figures show how molecular capture beads in a non-digitized sample will be contaminated from proteins or nucleic acids from many different cells the capture beads that have been compartmentalized with one cell will only capture molecules from that cell.

Thus, in comparing the singly functional approach to the method of FIG. 1; a large culture chamber cells would be producing cytokines and cytokine capture beads would capture proteins secreted by all the cells. However, if the cell is compartmentalized with the cytokine capture bead then only cytokines from that specific cell will bind. Similarly, as shown in FIG. 2b if cells are lysed in a large assay volume, a RNA capture bead would capture transcripts from all neighboring cells However, if the cell is compartmentalized before lysing then the capture bead binds only transcripts from the microdroplets.

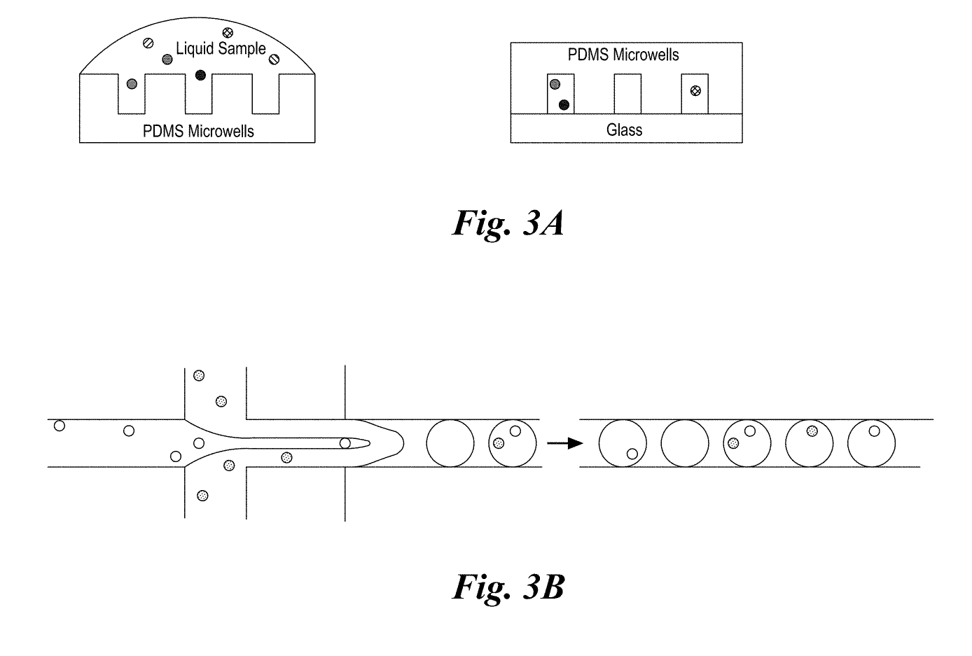

Limitations of current methods are further illustrated in FIGS. 3a and 3b whereby there is a challenge in using current microfluidic digital or single cell assays, with single function beads. FIG. 3a shows an example of a droplet based assay and FIG. 3b shows an example of a microwell platform. During compartmentalization or digitization both cells and beads are loaded randomly, and loading is governed by Poisson statistics; a Poisson distribution based on cell/bead dilution provides a framework to predict the number of microfluidic compartment with cells, beads, or those co-occupied by both a cell and bead; such as the Drop-seq assay (McCarroll Lab, Boston Mass.).

More specifically, as shown in FIGS. 3a and 3b, despite the utility of microfluidic digitization, microfluidic platforms remain limited by non-deterministic loading into the chambers. FIG. 3A shows in droplet based platforms cells and beads enter the droplet generator according to Poisson statistics and the dilution status of the sample. This results in a statistical distribution (Poisson) of droplets that are 1) empty, 2) contain just a capture bead, 3) contain just a cell, 4) contain the desired one cell and one bead, or 5) contain multiple cell/bead mixtures. FIG. 3a shows this effect in loading microchambers while FIG. 3b shows this effect in loading microdroplets. As shown the “Poisson” distribution of loading is true across microfluidic digitization platforms, including simple microwells, in which cells and beads settle or are pulled into an array of wells prior to microwell sealing and digitization.

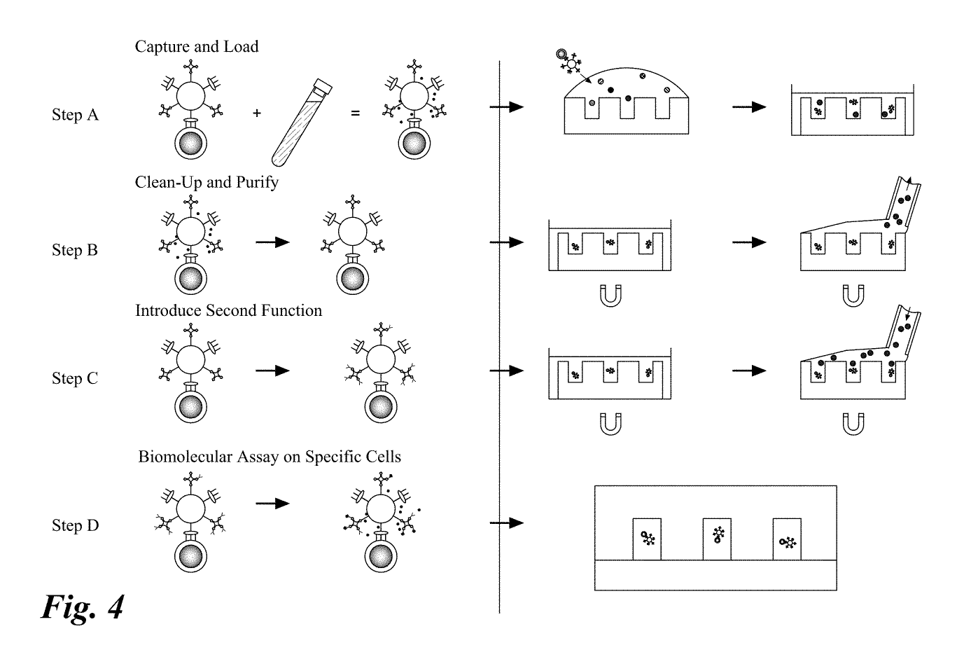

As shown in FIG. 4, in certain embodiments, a multifunctional bead may be used to remove the current limitation of digital microfluidic platforms. In certain embodiments, this includes the use of capture beads in the bulk solution, prior to digitization, for cell capture and then deterministic loading, for example. cells in each well. In other embodiments, it may be used for cleaning and purifying the cell once trapped in the microfluidic compartment. In yet another embodiment, may allow introduction of new reagents to the microfluidic compartment, such as the cytokine antibody in our example, and/or compartmentalization and initiation of the single cell or digital assay, such as cytokine capture in one example, including proteins and nucleic acids that interact with cell surface or intracellular molecules. In this case, the multifunctional bead may be used to retain the cell during multiple washing and binding steps, where a number of cell labels are being added to the chamber sequentially.

Therefore, as shown in FIG. 4, using a multi-functional bead during compartmentalization and digital loading within single cell microfluidic platforms may greatly expand their capability. First, the bead may be introduced before compartmentalization, into the bulk sample, for cell capture. This is possible, as the second function, biotinylated antibody in our example case, has not yet been added to bead. Unlike the singly functionalized bead shown in FIGS. 2a and 2b, contamination will not occur. The bead itself is now available for use in single cell compartment. The bead may be magnetic, or have other enabling features, that enable use in loaded the microfluidic wells/compartments.

Thus, as further illustrated in FIG. 4, in certain embodiments, the method comprises capturing cells from a bulk sample and loading into a digital device (step A), As illustrated in this example, the method may be used in a single cell cytokine secretion assay.

In certain embodiments, the bead may be a plastic microsphere coated with the multifunctional agents and may comprise polystyrene, latex beads, spheres or microspheres. In certain preferred embodiments, the bead may be a magnetic bead, which may be used to pull cells into different compartments or microwells of the device. This may further serve to hold the trapped cell in the well during a purification step; such as washing away contaminants prior to covering the well and digitization. The ability to hold the bead in the well, may now allow for the introduction of additional reagents and buffers; such as in the example shown.

In other embodiments, the bead may be a plastic microsphere or any solid support surface having a particle type shape. In certain embodiments, the microspheres may be particles between 0.1 and 100 μm in size. The size thus providing for a large surface-to-volume ration. They may be a made with a variety of materials providing that the surface may be functionalized. The bead materials may include, but are not limited to ceramics, glass, polymer, metals, or a combination thereof. In certain embodiments, the polymer may be polyethylene, polystyrene. In other embodiments, the metal may have magnetic properties.

In certain other embodiments, digitization can be complete, by closing off the microfluidic compartment in the microwell case, and the single cell/digital assay can commence. For example, the capture of secreted cytokines or cell lysis and capture of internal nucleic acids or proteins. Closing off the microfluidic compartment may be accomplished through formation of droplets, as shown in the droplet based examples, or by covering the compartments with a layer of oil and isolated the single cells in aqueous chamber, as in the microwell examples.

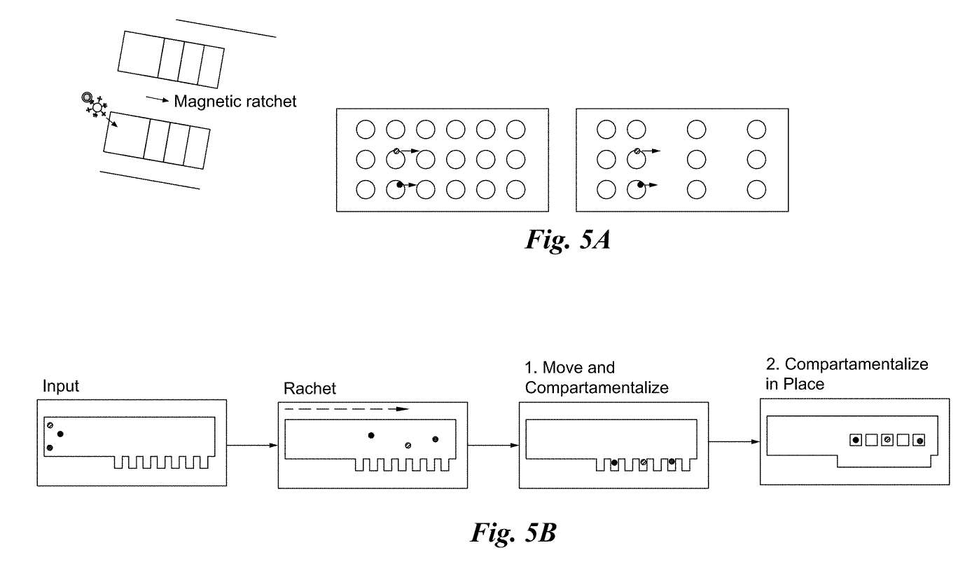

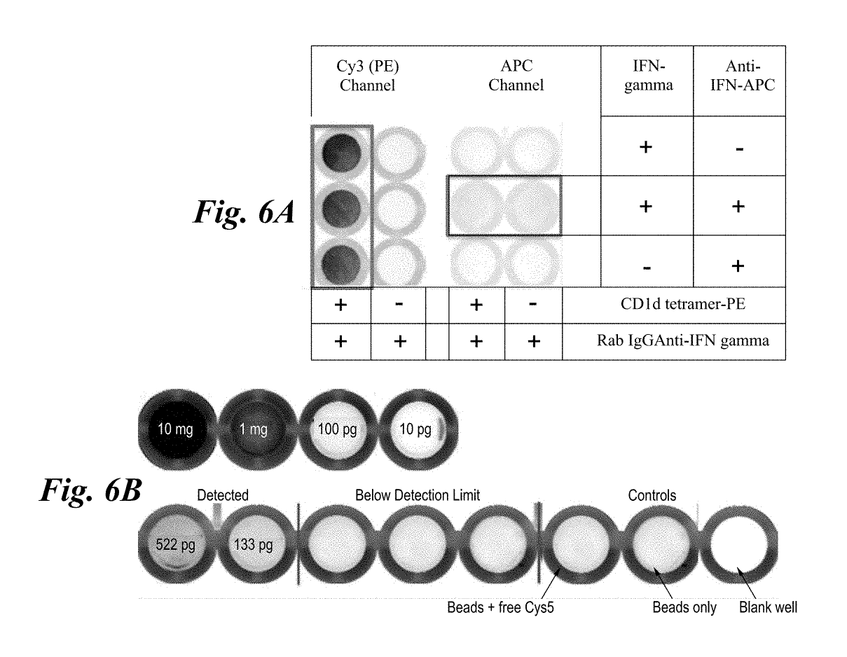

As shown, FIGS. 5a and 5b are illustrated examples of digital microfluidic platforms and multi-functional beads and the use of the device in the method described. As illustrated in FIG. 5a, a magnetic ratchet is used to separate cells from a bulk sample and trap them individually on magnetic pillars. The description of the use of a rotating magnetic field to actuate cells across the magnetic pillars, referred to as magnetic ratcheting allows movement of the cell with the multifunctional bead across the micropillar array.

FIG. 5b, shows the process steps, whereby, the cells are either isolated in place, for example by placing a microchamber array over the top of the device or by moving the cells using the magnetic ratchet to bring cells to an isolation chamber. For example, in one embodiment fluidic/cell isolation may be accomplished by running an oil phase across the isolation chamber array.

Thus, in one exemplary embodiments a specific multifunctional bead may be used for data collection, labelled with a cell capture (cD1d tetramer) and cytokine capture (IFN-gamma antibody) element. The multifunctional bead may also be simultaneously conjugated with an anti-PE antibody, enabling binding to the cD1 tetramer PE cell capture element, and an anti-igG antibody. The anti-igG antibody, enables binding to the antibody for cytokine capture. This produced a multifunctional bead capable of NKT cell capture and simultaneous detection of IFN-gamma.

In certain embodiments of the invention a using multifunctional beads with different functionality/utility may be used in collecting single cell data. For example, a multifunctional bead set containing a tetramer based cell capture element and a cytokine antibody for capturing secreted proteins. In an alternative example a multifunctional bead set containing an antibody based cell capture element and a cytokine antibody may be used for capturing secreted cytokines. In this embodiment, the bead is conjugated to the cell capture element, but the cytokine capture antibody (the second function) is introduced through adhesion to the cell surface. In another embodiment, a multifunctional bead set containing an antibody based cell capture element and a nucleic acid or oligonucleotide molecular capture element may be used.

More specifically a bead may be a specific tetramer used as the capture elements to the bead. For example, in one embodiment, biotinylated CD1D tetramers (Proimmune Ltd., Oxford, UK), specific for NKT cells were conjugated to streptavidin conjugated Dynabeads® (ThermoFisher Scientific, Pittsburgh Pa.) using manufacturer's specifications. The binding reaction was performed in the presence of an equal molar ratio of biotinylated cytokine specific antibodies (IFN gamma).

In an alternative embodiment, unlike the tetramer described above, the cell capture element may be an antibody for a cell surface protein marker. The second molecular binding element may be captured with the cell through binding to a different portion of the bead-cell complex; for example, the cell surface as illustrated.

In another embodiment, the molecular capture element may be a nucleic acid or oligonucleotide. Once captured in the microfluidic or digital compartment, the cell may be lysed, releasing its nucleic acid components for capture on a multifunctional bead. This can then be used in downstream nucleic acid amplification reactions and analysis.

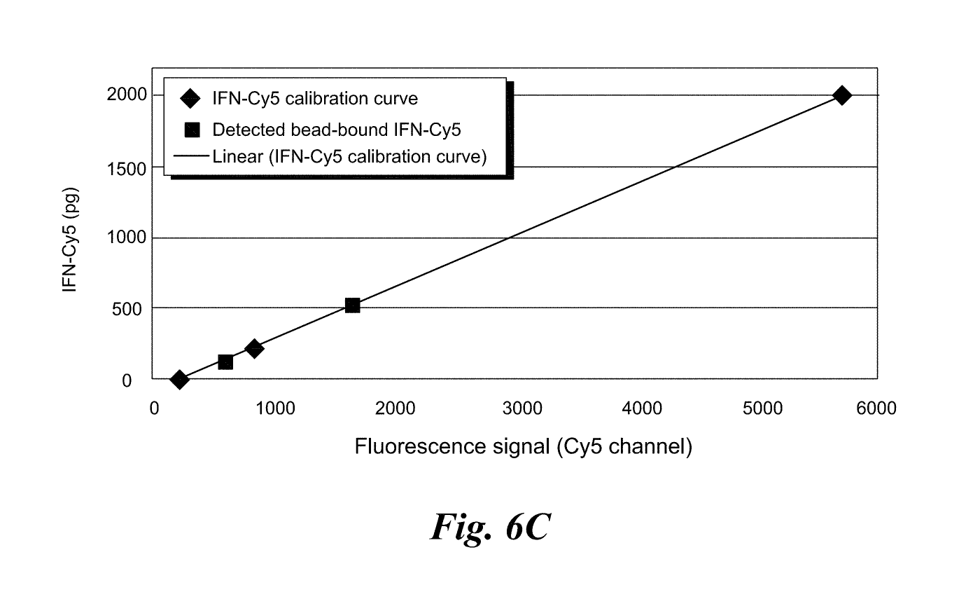

FIGS. 6a-c are representative data showing cytokine binding function on a bead conjugated with a pMHC II tetramer cell capture element and a cytokine binding antibody; an example of a multifunctional bead described above.

As shown in FIG. 6a, to test the utility of the multifunctional beads, the beads were then incubated with recombinant IFN gamma and a secondary antibody against IFN gamma (labelled with APC). The beads were imaged using a typhoon scanner with and without the CD1d tetramer and with and without the IFN gamma and IFN gamma antibody. The images of the reaction wells show that the cytokine capture antibody functioned with or without the additional CD1d tetramer function. Results showed equal fluorescent levels in wells singly labelled with ab or labelled with both ab and tetramer. It was found that the antibody capture remains specific on the multifunctional beads and there is no additional non-specific binding on the tetramer-labelled beads.

FIG. 6b shows further function of the cytokine capture element in binding recombinant cytokine (IFN). The top row of images is a calibration standard, where a serial dilution of the recombinant cytokine, and secondary, fluorescently labelled antibody for the cytokine, was added to an equal volume of multifunctional beads. The images show that with an increasing concentration of cytokine, the fluorescent signal from the bead population increasing. These results were taken using a Typhoon fluorescent scanner using the laser and fluorescent filters chosen for the IFN-APC conjugated secondary antibody. The bottom row of FIG. 6b is experimental data, were different test concentrations of cytokine were applied to the multifunctional beads and compared to the calibration data generated above. FIG. 6c, shows graphically the results of the cytokine binding test with a lowest measurable value occurring when 133 picograms (pg) of cytokine were added to the reaction per bead. This level of cytokine binding, in. pictograms, is consistent with an amount secreted by a single cell.

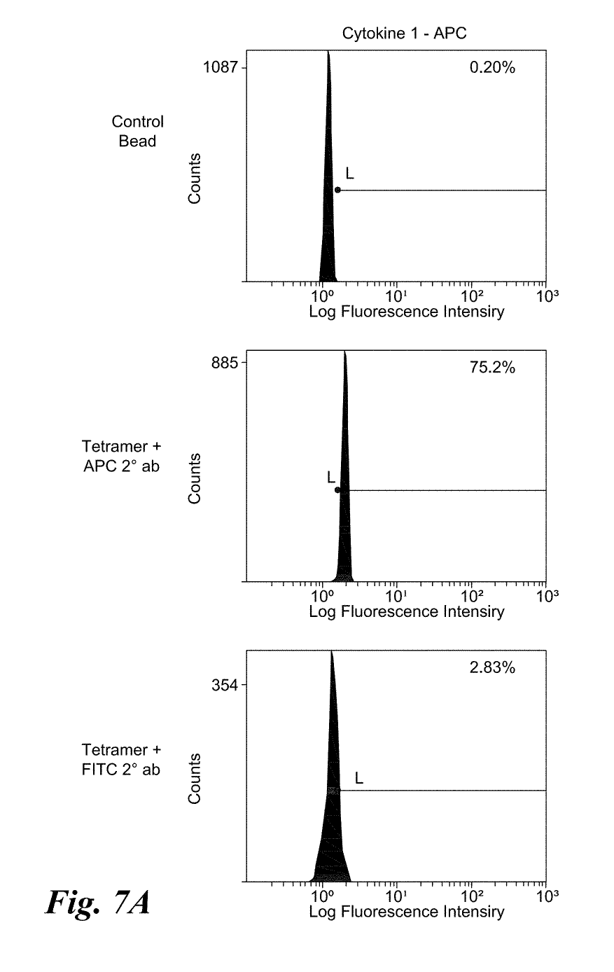

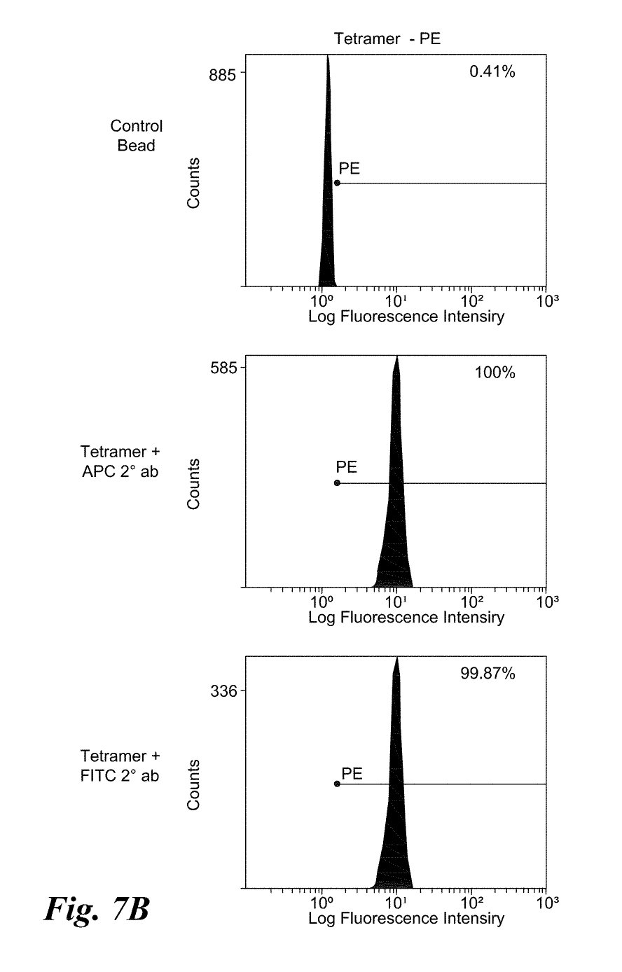

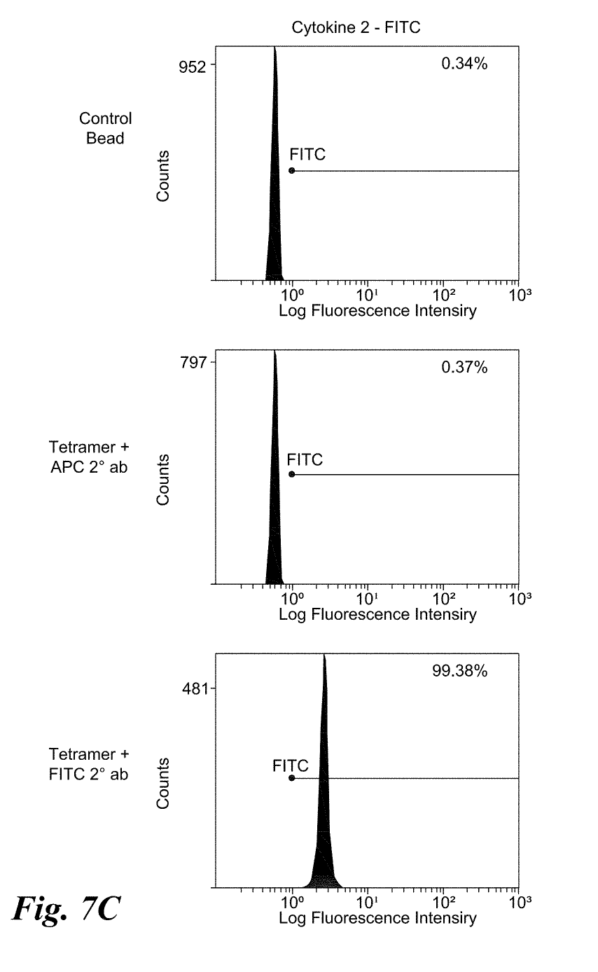

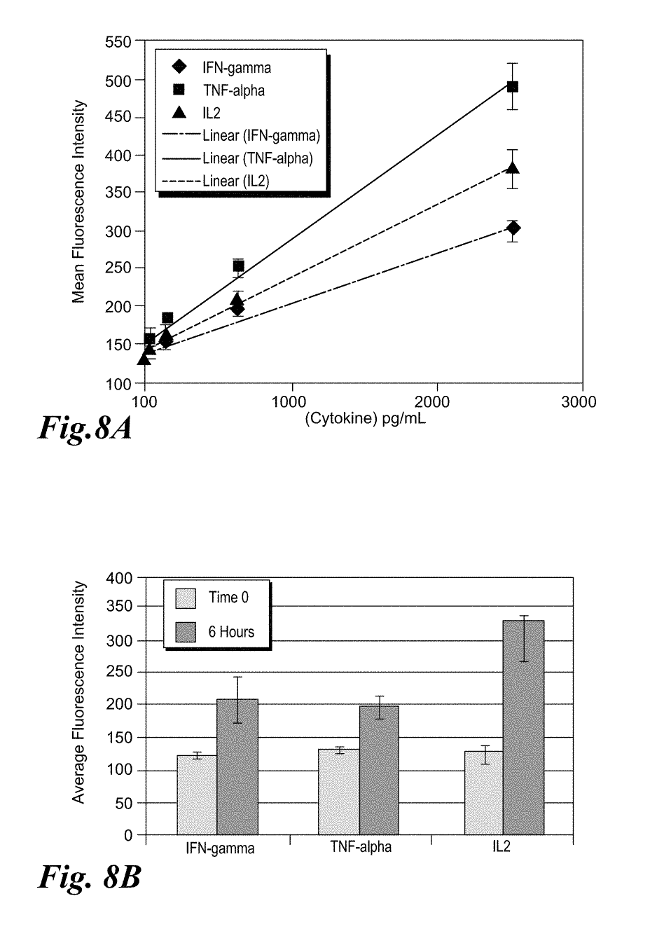

FACS or fluorescent characterization of individual multifunctional beads is also possible, as opposed to the bulk characterization shown above using the typhoon fluorescence scanner. This is shown in FIGS. 8a-8c which are histograms of bead fluorescence intensity, in the APC, PE, and FITC channels respectfully, of a BD flow cytometer (y-axis is number of counts/beads and x-axis is log fluorescence intensity). In each of the histograms, FIGS. 7a-c, row A are control beads showing the background fluorescence intensity before conjugation and incubation; the bar shows the cutoff level utilized for background subtraction in B and C. Row B are histograms from multi-functional beads conjugated with an IFN-gamma antibody and incubated with IFN and an APC-labelled secondary (2°) antibody. The graph shows clear signal from the beads in the PE channel, showing successful tetramer conjugation, and the APC channel, showing successful cytokine capture. Row C are additional histogram showing the same multi-functional beads, but labelled with a FITC secondary antibody after incubation with the cytokine showing the capability to perform multiplexed assays.

FIGS. 8a and 8b, depict data using the beads for cytokine binding after cell capture within microwells. Recombinant cytokines were first utilized to construct a standard curve, showing the limiting concentration detectable by the bead (A), and then the standard curve was used to estimate the amount of cytokine secretion after 6 hour incubation within microwells. The experiment was performed for three different cytokines (IFN-gamma, TNF-alpha, and IL2).

More specifically, FIG. 8a are T cells pre-activated with CD3/CD28 beads captured inside microwells, with the cytokine binding/multifunctional beads, and incubated with different levels of recombinant cytokine (IFN-gamma, TNF-alpha, IL10). The limit of detection of the captured cytokine binding beads was estimated by determining the lowest concentration that produces cytokine mediated fluorescence intensity three standard deviations beyond the no cytokine/blank control wells. Images were taken using a standard fluorescence microscope and the appropriate filters sets for the FITC, PE, and APC labels on the secondary antibodies, specific for each cytokine. Single cell/beads were digitally selected using a stand ImageJ particle counting script, and the average intensity of each bead was captured for the analysis.

FIG. 8b shows further data using T cells pre-activated with CD3/CD28 beads and captured inside microwells within beads. The cells were then incubated and allowed to secrete cytokines. The data shows the variation in cytokine secretion for activated T cells captured with the cytokine binding beads after a 6-hour incubation, the bead is capable of binding secreted cytokines and that if incubated in the presence of a secondary antibody the fluorescence around the bead (due to cytokine binding) becomes distinguishable from background fluorescence. The fluorescence intensities of the beads after incubation suggest secretion of 100-200 picograms of cytokine per cell within the six-hour period.

### EXPERIMENTAL

Various methods may be used to prepare multi-function beads for cell capture and cytokine analysis. In certain embodiments, the following method may be used.

Magnetic beads decorated with antibodies for both specific cell capture (positive selection) and binding of cytokines secreted by the captured cell were prepared in the following manner: 1 μm diameter (Dynabeads® MyOne Tosylactivated, 65501, ThermoFisher Scientific, Waltham, Mass.), 2.7 μm diameter (Dynabeads M-270 Epoxy), or 4.5 μm diameter (Dynabeads M-450 Epoxy) activated beads were first diluted in pure water at a concentration of ˜4×10⁸ beads/mL, mixed vigorously (pulsed vortex), and quickly settled via placement on a permanent magnet (e.g. DynaMag-2 magnet, ThermoFisher, 12321D) for approximately 1 min. After removal of the supernatant, a mixture of secondary antibodies were added to the pelleted beads. This mixture consisted of 50 μg mouse IgG1 anti-PE antibody (BioLegend, San Diego, Calif. 408102) along with 50 μg unlabeled goat anti-rabbit IgG (Jackson Immuno Research, West Grove, Pa111-005-144) in 100 mM sodium borate, pH 8.5. A total solution volume of 500 μL was added per ˜2×10⁸ total beads. After thorough mixing, the samples were protected from light and further incubated at room temperature for 16-24 hours under 500 rpm agitation.

After the overnight binding of the secondary antibodies to the activated bead surface, samples were placed on the permanent magnet for 1 min. after which the supernatant was removed as before. Beads were then resuspended in wash/block buffer consisting of 0.1% human serum (HS) in PBS and incubated for 5 minutes at room temperature. The buffer was again removed after magnetic capture and followed by two additional rounds of washing with 0.1% HS/PBS. The washed and blocked beads were next incubated with primary capture antibodies. To 100 μL bead slurry were added 10 μL each of at least 1 μM PE-labeled cell capture antibody, such as anti-CD154 (5C8 clone, Miltenyi Biotec, Bergisch Gladbach, Germany 130-098-289) along with at least 1 μM cytokine capture antibody, such as anti-IFN-gamma (abcam, Cambridge, Mass., ab25101). Samples were then mixed thoroughly before room temperature incubation under darkness for 1 hour with 500 rpm agitation.

After removal of the unbound primary antibody mixture, bead washing proceeded as before (3×0.1% HS/PBS) before final resuspension in 0.1% HS/PBS for medium-term storage at 4° C. or immediate application to cell capture experiments. Confirmation of antibody immobilization to the bead surface was achieved via application of fluorophore-labeled secondary antibodies and analysis via standard flow cytometry protocols or fluorescence imaging (GE Healthcare Live Sciences, Marlborough, Mass., Typhoon®FLA laser scanner).

While only certain features of the invention have been illustrated, and described herein, many modifications and changes will occur to those skilled in the art. It is, therefore, to be understood that the appended claims are intended to cover all such modifications and changes as fall within the scope and spirit of the invention.

What is claimed is:

1. A method of performing an assay on rare cells captured from a biological sample comprising:
contacting a solution containing the biological sample with a multifunctional bead, the multifunctional bead comprising;
a microsphere between 0.1 and 100 μm in size;
a cell capture element, on the surface of the microsphere, capable of binding to a protein or cell specific marker on the surface of a rare cell; and
a biomolecular capture element, on the surface of the microsphere, capable of binding to biomolecular components contained within or produced by the rare cell;
incubating the multifunctional beads with a biological sample containing the rare cells and binding the multifunctional bead to the rare cells through the surface capture element to create a bead-bound rare cells;
capturing biomolecules contained within or produced by the rare cells through the biomolecular capture element; and
assaying the rare cells by analyzing the biomolecules captured by the biomolecular capture element.

2. A method of claim 1 where the cell capture element is a MHC tetramer capable of binding to antigen specific T cells.

3. The method of claim 2 where the capture element is an antibody capable of binding to a cell surface marker on the target cell.

4. A method of claim 1 where the biomolecular capture element, are antibodies, nucleic acids, oligonucleotides, artificially synthesized bioactive polymers, fluorescent conjugates, metal conjugates or a combination thereof.

5. The method of claim 4 where the biomolecular capture element is an antibody.

6. The method of claim 1 where the analyzing the biomolecules comprises, nucleic acid sequencing, cytokine secretion analysis, quantification of gene expression, quantifying the amount of rare cells in the biological sample, quantifying the functional activity of the rare cells in the biological sample or a combination thereof

7. The method of claim 1 where the analyzing the biomolecules comprises encoding and/or barcoding of selected rare cells.

8. The method of claim 1 further comprising the step of transforming the bead-bound rare cells into droplets prior to capturing biomolecules.

9. A method of capturing rare cells from a biological sample comprising:
contacting a solution containing the biological sample with a multifunctional bead, the multifunctional bead comprising;
a microsphere between 0.1 and 100 μm in size;
a cell capture element, on the surface of the microsphere, capable of binding to a protein or cell specific marker on the surface of a rare cell; and
a biomolecular capture element, on the surface of the microsphere, capable of binding to biomolecular components contained within or produced by the rare cell;
binding the multifunctional bead to the rare cells through the surface capture element to create bead-bound rare cells;
flowing the solution containing the bead-bound rare cells through a microfluidic device, the microfluidic device having microfluidic compartments;
partitioning the bead-bound rare cells of the solution into at least one of the microfluidic compartments;
contacting a biomolecular capture element with bead-bound rare cells; and
capturing biomolecules contained within or produced by the rare cells through the biomolecular capture element.

10. A method of claim 9 where the cell capture element is a WIC tetramer capable of binding to antigen specific T cells.

11. The method of claim 10 where the capture element is an antibody capable of binding to a cell surface marker on the target cell.

12. A method of claim 9 where the biomolecular capture element is antibodies, nucleic acids, oligonucleotides, fluorescent conjugates, metal conjugates, artificially synthesized bioactive polymers or a combination thereof.

13. The method of claim 12 where the biomolecular capture element is an antibody.

14. The method of claim 9 further comprising the step of assaying the rare cells by analyzing the biomolecules captured by the biomolecular capture element.

15. The method of claim 14 where the analyzing the biomolecules comprises nucleic acid sequencing cytokine secretion analysis, quantification of gene expression, a, quantifying the amount of rare cells in the biological sample, quantifying the functional activity of the rare cells within the sample or a combination thereof

16. The method of claim 14 where the analyzing the biomolecules comprising encoding and/or barcoding of selected rare cells.

17. The method of claim 9 where the multifunctional bead is magnetic and partitioning the bead-bound rare cells comprises magnetic trapping of the bead-bound rare cells on magnetized pillars of the microfluidic compartments.

18. The method of claim 9 where the multifunctional bead is placed in the digital microfluidic device prior to contact with the biological solution.

19. The method of claim 18 where one or more of the microfluidic compartments contain a biomolecular capture element.

20. The method of claim 19 where different biomolecular capture elements are contained in different microfluidic compartments.

21. A multifunctional bead for capturing rare cells, the multifunctional bead comprising;
a microsphere between 0.1 and 100 μm in size.
a cell capture element, on the surface of the microsphere, capable of binding to a protein or cell specific marker on the surface of a rare cell; and
a biomolecular capture element, on the surface of the microsphere, capable of binding to biomolecular components contained within or produced by the rare cell.

22. The multifunctional bead of claim 21 where the microsphere is ceramics, glass, polymer, metals, or a combination thereof.

23. The multifunctional bead of claim 22 where the microsphere is polyethylene, polystyrene, or a combination thereof.

24. The multifunctional bead of claim 21 wherein the microsphere is magnetic.

25. The multifunctional bead of claim 21 where the cell capture element is a MHC tetramer capable of binding to antigen specific T cells.

26. The multifunctional bead of claim 21 where the capture element is an antibody capable of binding to a cell surface marker on the target cell.

27. The multifunctional bead of claim 21 where the biomolecular capture element is antibodies, nucleic acids, oligonucleotides, fluorescent conjugates, metal conjugates, artificially synthesized bioactive polymers or a combination thereof.

28. The multifunctional bead of claim 21 where the bead is a magnetic microsphere, the cell capture element is a WIC tetramer, and the capture element is an antibody.
